(19) United States
(12) Patent Application Publication
(10) Pub. No.: US 2008/0004237 A1
(43) Pub. Date: Jan. 3, 2008

(54) Polysacchocride prodrug of 5-fluorouracil (5-FU) with enhanced target specificity for galectin-3 expressing cancers

(76) Inventors: Joemy C. Tam, Medford, NY (US); QiBing Mei, Xian (CN); Dale K.M. Choi, Medford, NY (US)

Correspondence Address: Joemy C. Tam, Suite 2, 3235 Route 112, Bldg. 5, Medford, NY, 11763, US

(21) Appl. No.: 11/657,754

(22) Filed: Sep. 17, 2007

(30) Foreign Application Priority Data
Feb. 23, 2006 (CN) 200610041838.0

(51) Int. Cl. A61K 31/716; C08B 37/00; C12P 19/04

(52) U.S. Cl. 514/54; 435/101; 536/18.7

(57) ABSTRACT

This application discloses embodiments of a novel prodrug and its method of synthesis. The prodrug comprises a galactose-containing polysaccharide covalently linked to 5-fluorouracil (5-FU). The galactose residues that are part of the backbone of the galactose-containing polysaccharide mediate the binding between the prodrug and the lectin galectin-3 which is expressed in various cancers. The galactose-containing polysaccharide is isolated from various plant material and covalently bonded to 5-FU. Various formulations (parenteral, or other local or systemic forms) can be used to administer this 5-FU-releasing prodrug to target galectin-3 expressing cancers.

Throughout this application, references are made to various publications. Disclosures of these publications in their entireties are hereby incorporated by reference into this application to more fully describe the state of the art to which this invention pertains.

### FIELD OF THE INVENTION

This invention involves drugs for the treatment of galectin-3 expressing cancers and their preparation methods, which mainly concern a kind of prodrug composed of the anticancer drug 5-fluorouracil (5-FU) and polysaccharides containing galactoses and its preparation methods.

### BACKGROUND OF THE INVENTION

The galectins are a family of lectins found in humans and other animal species. Lectins (also called carbohydrate binding proteins) recognize specific oligosaccharide structures on glycoproteins and glycolipids. Members of the galectin family have been suggested to mediate cell adhesion, regulate cell growth and apoptosis (Perillo et al 1998).

Galectin-3, a 31 kDa member of the β-galactoside-binding lectin family, is an intra- and extra-cellular lectin which interacts with extracellular matrix proteins, cell surface molecules and intracellular glycoproteins. It is expressed in epithelial and immune cells. It has multiple biological functions including cell growth, adhesion, differentiation, proliferation, angiogenesis, and apoptosis (Perillo et al 1998, van de Brule et al 2002, Califice et al 2004).

Recent research revealed that Galectin-3 is associated with processes that effect or regulate cancer invasion and metastasis, such as angiogenesis, cell-matrix interaction, dissemination through blood flow and extravasation. Its expression was found to correlate with cancer progression, metastasis, and therefore, may have prognostic value. Increased expression of Galectin-3 have been found in breast, lung, prostate, bladder, thyroid, other head and neck, lymphoma, colon, pancreas and other gastrointestinal cancers (Schoeppner et al 1995, Le Marer et al 1996, Moon et al 2001, Takenaka et al 2002, Yoshii et al 2002, Buttery et al 2004, Teymoortash et al 2006). The mechanisms by which galectins exert these diverse effects remain largely unknown.

5-fluorouracil (5-FU) is a well-established chemotherapeutic agent available for many decades. It is a pyrimidine analog and belongs to the class of anti-metabolites. It is effective in many types of cancer, including breast, colorectal, other gastrointestinal malignancies and skin cancers. Once administered to the patients, 5-FU tends to distribute widely in the body with low selectivity thus results in significant toxicities. If an appropriate carrier is chemically linked to the 5-FU with specificity for galectin-3 expressing cancers cells, this will enhance the therapeutic index as well as allow the dosage of the drug to be reduced to minimize toxicities. With such design, the 5-FU prodrug can have selectively against galactin-3, thus allowing preferential binding to galectin-3 expressed cancers to maximize efficacy. Various formulations (parenteral, or other local or systemic forms) can be used to administer this 5-FU prodrug to reach the target cancers and exerts its efficacy.

Carriers include ethylene or acrylic acid polymers, polysaccharides, hydroxyl-acid polymers, and amino acid polymers, etc. Ouchi and others reported 4 kinds of conjugations composed of 5-FU and chitosan through ester linkage, amino formamide, amide linkage and ether linkage (Ouchi et al, 1992). Ohya and coworkers reported a conjugate of 5-FU and 6-O-carboxymethyl chitosan, which has an inhibitory effect on P388 lymphoid leucosis (Ohya et al 1992, 1993). Fan and colleagues composed a co-polymer of lactic acid-phosphate ester with 5-FU as the model drug in the side chain and the copolymer of lactic acid-phosphate ester as the large molecule prodrug carrier, which shows lower toxicity and better antitumor activity (Fan et al, 1985; Luo et al, 1994). Zhu and others linked 5-FU with poly-L-(2-ethoxyl)-asparagine to make a large molecule prodrug, which shows improved drug release effect in rabbits and maintains a steady plasma concentration during release (Zhu et al, 2003).

The literature above described several possible means to prepare 5-FU prodrugs. However, the scope of these processes primarily focuses on the drug release pattern with no intention to specifically direct the 5-FU to the galectin-3 expressing cancer cells. For example, a slow-release preparation only prolongs the release time of the drug in the body and cannot direct the drug specifically to the target tissue of galectin-3 expressing cancer cells. A slow-release preparation is completely different from a targeting preparation in their administration mechanism, pharmacokinetics, efficacy and safety profile. Therefore, the current invention is not a repetition of these preparations.

Over the years, there are also a number of patents applied to other 5-FU or related subjects. The following is a list briefly describing these various patents, which have been applied to 5-FU or related matters in various aspects:
U.S. Pat. No. 4,605,738, issued to Kamata et al in 1986, discloses a process of producing 1-phthalidyl-5-fluorouracil derivative as an anticancer agent.
U.S. Pat. No. 4,622,325, issued to Fujii et al in 1986, discloses a formulation comprises a combination of 1-n-hexylcarbamoyl-5-fluorouracil and a uracil salt.
U.S. Pat. No. 4,631,342, issued to Umemoto et al in 1986, discloses a process for producing 5-fluorouracil using an aqueous phosphoric acid solution as a solvent.
U.S. Pat. Nos. 4,650,801 and 4,652,570, issued to Fujii et al both in 1987 respectively, discloses a formulation comprises a combination of a 5-fluorouracil derivative and a uracil derivative.
U.S. Pat. No. 4,704,393, issued to Wakabayashi et al in 1987, discloses a compound as 1-substituted 5-fluorouracil for use in inhibiting platelets aggregation.
U.S. Pat. No. 4,719,213, issued to Fujii et al in 1988, discloses a formulation comprises a combination of a 5-fluorouracil prodrug [as either 1,3-bis(2-tetrahydrofuryl)-5-fluorouracil or 3-(2-tetrahydrofuryl)-5-fluorouracil] and a uracil salt.
U.S. Pat. No. 4,757,139, issued to Kawaguchi et al in 1988, discloses a 5-fluoro-2′-deoxyuridine derivative with anti-tumor effect in low doses.
U.S. Pat. No. 4,810,790, issued to Fujii et al in 1989, discloses 5-fluorouracil derivatives of the formula: ##STR1## wherein R.sup.1 is a fluorine-containing C.sub.1-C.sub.10 organic group which optionally contains sulfur, oxygen and/or nitrogen, with usage as a carcinostatic substance.
U.S. Pat. Nos. 4,864,021 and 4,983,609, issued to Fujii in 1989 and 1991 respectively, discloses 5-fluorouracil derivative residue of the formula ##STR1## or ##STR2## using various chemical subgroups as substitutes for the R-component, which can be converted to 5-fluorouracil in vivo and is linked to the carbonyl part by an ester or amide linkage.
U.S. Pat. No. 4,914,105, issued to Fujii et al in 1990, discloses a formulation comprises a combination of a 5-fluorouracil derivative and a uracil derivative.
U.S. Pat. No. 5,032,680, issued to Kawai et al in 1991, discloses a 2′-deoxy-5-fluorouridine derivative, which exhibits anti-tumor activities with purported lower toxicity.
U.S. Pat. No. 5,047,521, issued to Fujii et al in 1991, discloses 5-fluorouracil derivatives represented by the formula ##STR1## or ##STR2## using various chemical subgroups as substitutes for the R-component, with the provision that R.sup.1 and R.sup.2 are not hydrogen atoms or specific acyl groups at the same time.
U.S. Pat. No. 5,049,551, issued to Koda et al in 1991, discloses a 5-fluorouracil derivative represented by the formula ##STR1##, ##STR2##, and ##STR3## wherein various chemical subgroups are used as substitutes for the R-component.
U.S. Pat. No. 5,077,055, issued to Muller et al in 1991, discloses a topical therapeutic system comprising 5-fluorouracil.
U.S. Pat. No. 5,089,503, issued to Johnson in 1992, discloses a temperature stable 5-fluorouracil formulation.
U.S. Pat. No. 5,116,600, issued to Fujii et al in 1992, discloses a composition and method for inhibiting inflammation caused by non-parenteral administration of 5-fluorouracil type compounds.
U.S. Pat. Nos. 5,457,187 and 5,663,321, issued to Gmeiner et al in 1995 and 1997, respectively, discloses oligonucleotides containing 5-fluorouracil exhibit antitumor activity and the synthesis and utilization method.
U.S. Pat. No. 5,496,810, issued to Schwartz in 1996, discloses a method of treating malignancies using a combination of 5-fluorouracil, alpha-interferon, and pyrimidine deoxyribonucleoside.
U.S. Pat. No. 5,610,160, issued to Sloan et al in 1997, discloses a topical 5-fluorouracil prodrug formulation and its preparation method.
U.S. Pat. No. 5,614,505, issued to Gmeiner et al in 1997, discloses a homo-oligomeric prodrug of 5-fluorouridine (5-FU) and 5-fluorodeoxyuridine, which is used as a polymeric drug delivery system for the production of FdUMP, the active inhibitor of thymidylate synthase.
U.S. Pat. Nos. 5,627,187 and 5,817,666, issued to Katz in 1997 and 1998, discloses a dermatologic formulary preparation of 5-fluorouracil with alpha hydroxy carboxylic acid for the treatment of actinic kerotoses.
U.S. Pat. No. 5,676,973, issued to Levin in 1997, discloses a topical formulation combining 5-fluorouracil and Live Yeast Cell Derivative (LYCD) for medical use.
U.S. Pat. No. 5,808,049, issued to Yamazaki, et al in 1998, discloses a method for preparing a stereospecific 5-FU ester compound that is resistant to decomposition in blood, but is quickly hydrolyzed in cancer cells.
U.S. Pat. No. 5,843,917, issued to Boyd et al in 1998, discloses compounds comprising 5-fluorouracil or 5-fluorouridine covalently linked to 5-ethynyluracil, 5-ethynyluridine or 5-propynyluracil for pharmaceutical use.
U.S. Pat. No. 6,403,569, issued to Achterrath in 2002, discloses a method for treating cancer using camptothecin derivatives and 5-fluorouracil
U.S. Pat. No. 6,670,335, issued to Singh et al in 2003, discloses a topical oil-in-water emulsion formulation contains 5-fluorouracil and 5-fluorouracil impregnated in porous microparticles.
U.S. Pat. No. 6,794,370, issued to Achterrath in 2004, discloses a method for treating metastatic colorectal cancer using CPT-11, 5-fluorouracil, and folinic acid as synergistic combination therapy.

None of the reference and patents mentioned above, taken either singly or in any combination, describes the present invention as claimed.

Accordingly, there is a need in the field to invent such a product. As can be seen from the data enclosed herein, this novel polysaccharide-based 5-FU prodrug possessing enhanced target specificity to galectin-3 expressing cancers cells. This unique property of the invention can lead to a higher efficacy and/or a reduced toxicity profile, thus providing a preferential method to deliver 5-FU to galectin-3 expressing cancers treatment.

### SUMMARY OF THE INVENTION

This invention utilizes prodrug technique to couple polysaccharides containing galactose with 5-FU through different bridging linkages to yield therapeutic conjugates. Because of this unique design, the 5-FU prodrug produced can have preferential binding to Galectin-3 and when appropriately administered to Galectin-3 expressed cancers, it can lead to maximal efficacy and reduced toxicity. Overall, using polysaccharide containing galactose as the carrier of 5-FU will have the targeting effect specifically at the galectin-3 expressing cancers cells, resulting in enhanced therapeutic effect of 5-FU. With increased selectivity and improved safety profile, dosage flexibility is feasible, allowing an oncologist to either push the 5-FU dose for maximal efficacy and/or reduce the 5-FU dose in frail or elderly patients to minimize toxicity. In addition, many polysaccharides also have immunoregulation function along with some anti-tumor effect. This may be able to help reduce the immunosuppression effect from 5-FU. This invention therefore combines the medical design concepts of drug delivery, targeting, and synergism to achieve the goal of high efficacy and low toxicity.

### BRIEF DESCRIPTION OF THE DRAWING

Other objects, features and advantages will be apparent from the following detailed descriptions of preferred embodiments taken in conjunction with the accompanying drawings in which:

FIG. 1. For illustration purposes only, an embodiment of the inventive drug delivery system wherein the galactose-containing polysaccharide is pectin, and Z refers to 5-FU. The symbols “**” and “*” indicate the position of β(1-4) glycosidic linkages, and n is from 1 to about 25,000.

### DETAILED DESCRIPTION OF THE INVENTION

The purpose of this invention is to provide a novel prodrug and methods of its preparation and, for the targeted delivery of galectin-3 expressing tumors, cells and tissues.

Definitions

The following terms are used as defined below. The use of these terms does not preclude the use of other terms not defined herein that are essentially synonymous with the defined terms.

The term prodrug refers to a compound whose efficacy is greatly enhanced after a conversion step that occurs in vivo after administering the compound to a subject or patient.

The term galactose-containing polysaccharide refers to a polysaccharide having at least one galactose residue. A galactose-containing polysaccharide may be naturally occurring or may be prepared by modifying a different polysaccharide. Further, a galactose-containing polysaccharide may comprise unmodified galactose residues or modified galactose-derived residues.

The term galactose-containing fragment refers to a portion of the galactose-containing polysaccharide that may arise from being acted on by various enzymes. Enzymes that will generate galactose-containing fragments are largely expected in the colon. These enzymes are largely bacterial in nature.

The term therapeutic parent compound refers to a compound having therapeutic and/or diagnostic properties in a form prior to its linkage to a galactose-containing polysaccharide. The term parent compound is generally a synonym.

The term derivatize or derivatizing refers to modifying a compound, e.g., galactose-containing polysaccharide or a therapeutic parent compound, by adding one or more reactive groups to the compound by reacting the compound with a functional group-adding reagent. As used herein, the term also refers to the attachment of cross-linkers to the compounds. The cross-linkers may be bifunctional, thus reacting with both compounds. A cross-linker possesses spacer arms that vary in size in different cross-linking compounds. This may be useful if one elects to have a known fixed distance between the galactose-containing polysaccharide and therapeutic parent compound.

The term linkage or linking bond refers to the covalent bond connecting, or linking, the galactose-containing polysaccharide and the therapeutic parent compound. This bond may be formed by attaching one or more functional groups to either of, or both of, the therapeutic parent compound and galactose-containing polysaccharide. The galactose-containing polysaccharide and/or the therapeutic parent compound may be derivatized by addition of the functional groups.

The term conjugate as used herein refers to the prodrug of the structural formula polysaccharide-R-Z.

The term targeting or targeted refers to the preferential distribution of the prodrug to a galectin-3 expressing cell, tissue or tumor, when compared to galectin-3 nonexpressing cell, tissue, or tumor.

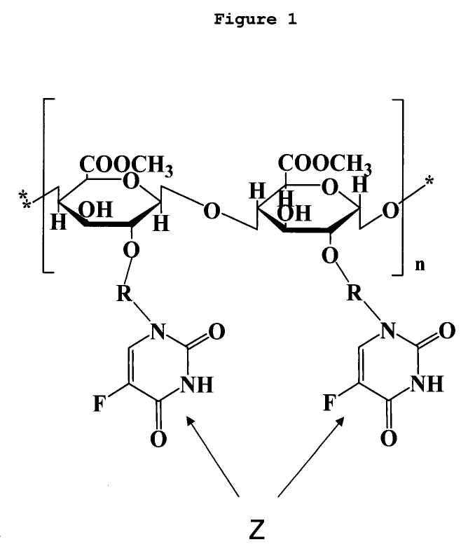

The purpose of this invention is to develop a novel prodrug for the targeted treatment of galectin-3 expressing cancers as indicated in FIG. 1 (5-FU prodrug for short), and its preparation methods. This design can increase 5-FU selectivity, enhance its therapeutic effects, and reduce toxicities.

The technical proposal of this invention is a prodrug for the targeted treatment of galectin-3 expressing cancers and the prodrug's methods of preparation having structure shown FIG. 1. The structure shown is the prodrug for the treatment of the galectin-3 expressing cancers resulted from the linkage of polysaccharides with 5-FU through various bridge linkages. Several features of the prodrug are:
The polysaccharides are galactose-containing polysaccharides.
The polysaccharides with galactoses may be purified from natural gums and plants.
The natural gums are pectin, guar gum, and carob bean gum, and the plant polysaccharides include aloe polysaccharide, medlar polysaccharide, and rhubarb polysaccharide.
The prodrug's structure may be represented as Polysaccharide-R-5-FU, in which R can be replaced with any one of the following functional groups: R═—(CH₂)ₙ—, —CO—, —CO(CH₂)ₙ—, and —CO(CH₂)ₙ—CO—, wherein n is from 1 to 4.
The pectin, guar gum, and carob bean gum are hydrolyzed first with alkali (pH=9−10) then with acid (pH=3−5), and precipitated in alcohol and dialyzed to produce natural gums of target molecular weights of approximately 10⁵ to approximately 10⁷ Da.

The extraction method for the aloe polysaccharide, medlar polysaccharide, and rhubarb polysaccharide is as follows: First, pulverized the aloe/medlar/rhubarb plant material, and boiled with ethanol for three eight-hour-periods. The components dissolved in ethanol are extracted. The residue is boiled with water for another three eight-hour-periods in order to extract polysaccharides. All the water extractions are then collected. The polysaccharide-enriched fractions are obtained by precipitation with 5 volumes of ethanol for 3 times. After removing proteins by dialysis, separate and purify with gel filtration chromatography, polysaccharide components are obtained with molecular weights of 10⁵-10⁷ Da.

During the extraction process, the following analytical instrumentation and techniques are implemented: a). High-performance liquid chromatography (HPLC) for purity analysis; b). Ultraviolet (UV) and infrared spectroscopic identification for qualitative examination; c). Measurement of sugar and glycuronic acid contents respectively by vitriol-phenol and vitriol-carbazole methods; and d). Measurement of the monosaccharide compositions of the polysaccharides of different molecular weights and their component ratio was performed by chromatographic techniques and gas chromatography. An illustrative alternative embodiment of a method for linking the galactose-containing polysaccharide to Z is:
1. Modifying the free hydroxyl group of a galactose residue to a reactive carboxyl group (e.g. an acyl chloride).
2. The product of step 1 can react with a —NH₂ or similar functional group (e.g., .—NHNH₂, —ONH₂, —NHC═(O)NHNH₂, —OH, —CO₂H, or —SH) of a therapeutic parent compound, with the modified carboxyl group of the galactose residues contained in the polysaccharides via the formation of an ester, ether, amide or acyl amine, a thioester or a thioether.

The following is but one illustrative embodiment of a method for linking the galactose-containing polysaccharide with a parent therapeutic compound, Z. A hydroxyl group in the 2 position of galactose is activated by chloroacetic acid. Then the activated carbonyl linker group reacts with the amine group of Z. This synthesis is exemplified below, using 5-FU as the embodiment of Z.

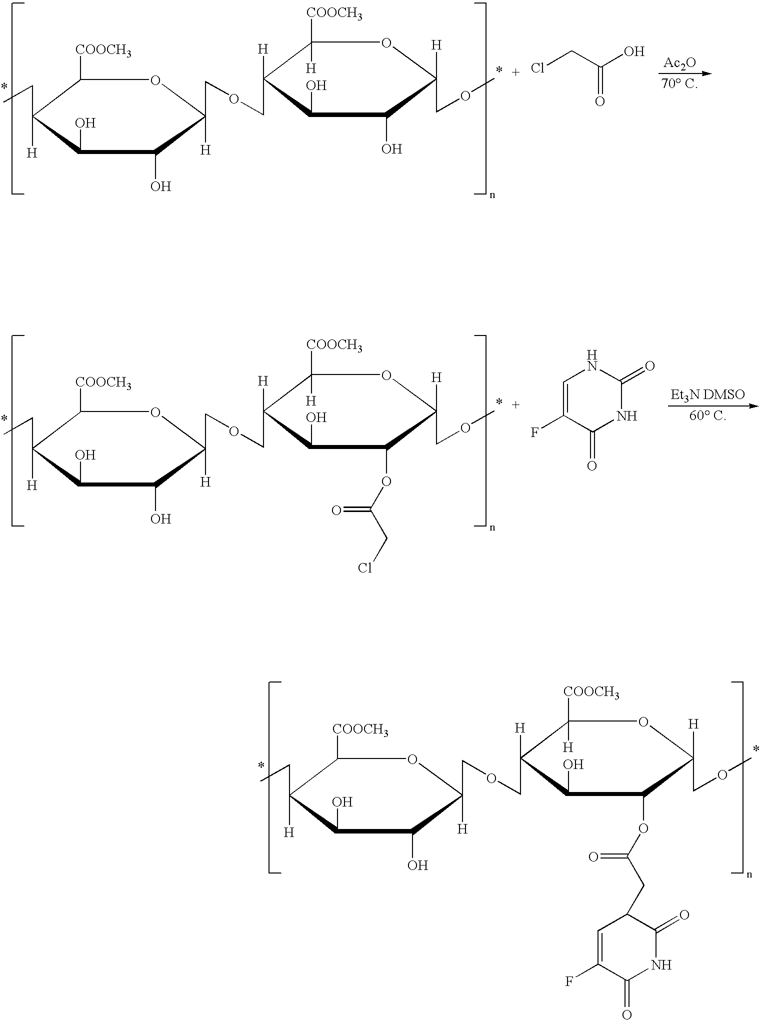

Alternatively, an amine group in an appropriate embodiment of Z reacts with chloroacetic acid to create a carboxylic acid linker group. Subsequent DCC (dicyclohexylcarbodiimide) coupling will link Z's newly added carboxylic acid linker group to the hydroxyl group in the 2 position of galactose in a galactose-containing polysaccharide. This method of linking is shown below.

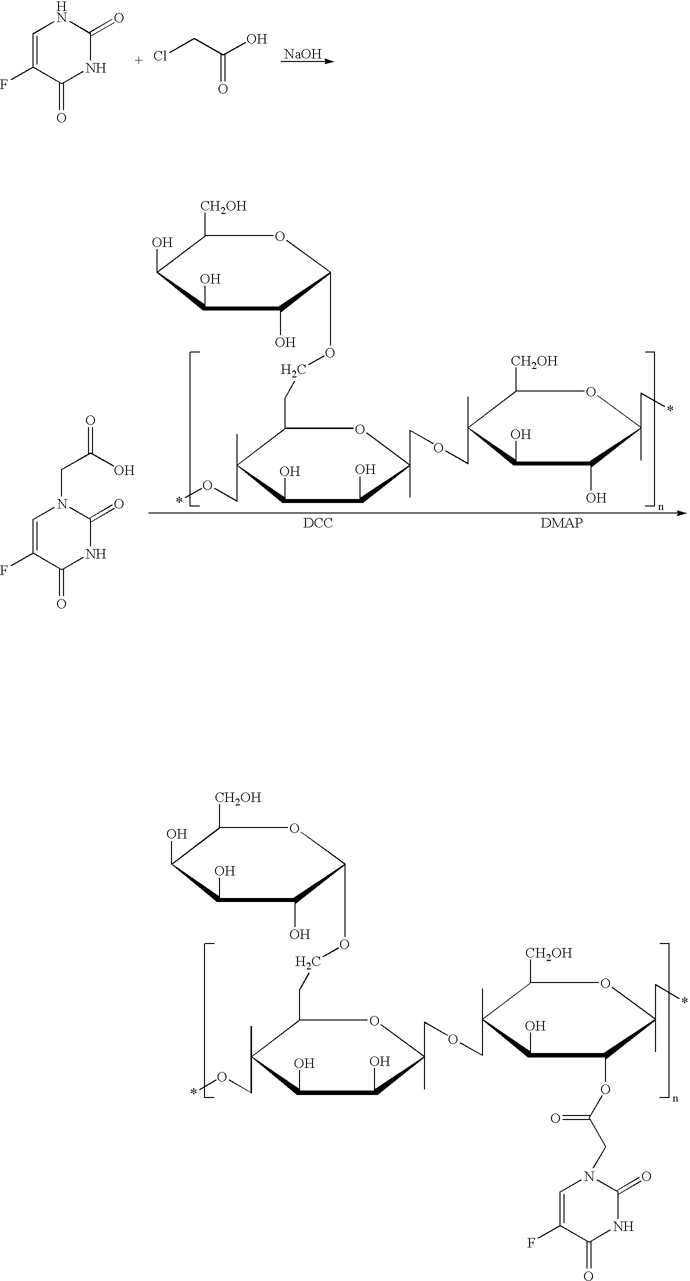

The formation of the ester linkage is made through the acyl chloride method or N,N′-dicyclohexylcarbodiimide (DCC) method. The formation of the ester linkage is carried out through condensation. The formation of the acyl-amine linkage is derived from aminolysis of acyl chloride.

For illustration purposes a nonlimiting embodiment of the inventive drug delivery system is described below.

Pectin: a polysaccharide composed of straight chains of galacturonic acid. The symbols “**” and “*” indicate the position of β(1-4) glycosidic linkages, and n is from 1 to about 12,500.

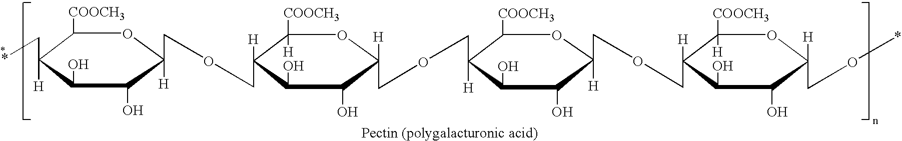

Guar gum: a non-ionic polysaccharide mainly polymerized with galactose and mannose, belonging to natural galactomannan with mannose as its main chain and β(1-4) glycoside link as the linkage between D-mannopyranose units. Meanwhile, galactopyranose is connected to the mannose main chain through α(1-6) link. The molar ratio between mannose and galactose is 2:1.

Carob bean gum: a colorless and flavorless polysaccharide refined from plant endosperm, mainly containing mannose and galactose with an average molecular weight of 300 kDa.

It is currently known that the natural occurring gums containing galactose residues, such as pectin, guar gum, and carob bean gum have the functions of regulating the bacterial colonies in the intestinal tract as well cholesterol lowering. In addition, aloe polysaccharides, medlar polysaccharides, and rhubarb polysaccharides are rich in galactose with known immunoregulation functions, which have not, as of yet, been fully explored for pharmaceutical development.

### Preparation of the Prodrug for Targeted Treatment of Galectin-3 Expressing Cancers

The preparation methods of the prodrug involved in this invention and the release of a therapeutic parent compound (exemplified by 5-FU) at the target site will be described below. It should be appreciated that the scope of the invention is not limited to the examples described below. Virtually any therapeutic parent compound that can be linked with a galactose-containing polysaccharide by one of the covalent linkages disclosed herein, is a suitable candidate for Z.

### Preparation Method of the Novel Family of Prodrugs for the Targeted Delivery to Galectin-3 Expressing Tumors, Cells and Tissues

The natural gums containing galactose residues are hydrolyzed first with alkali (pH=9-10) then with acid (pH=3−5), and precipitated with alcohol and dialyzed to obtain natural gums of targeted molecular weights (10⁵-10⁷ Da) containing galactose residues.

The extraction method for galactose such as aloe polysaccharide, medlar polysaccharide, and rhubarb polysaccharides is to pulverize the aloe/medlar/rhubarb plant material, and boiled with ethanol for three eight-hour-periods. The components dissolved in ethanol are extracted. The residue is boiled with water for another three eight-hour-periods in order to extract polysaccharides. All the water extractions are then collected. The polysaccharide-enriched fractions are obtained by precipitation with 5 volumes of ethanol for 3 times. After removing proteins, dialysis, separate and purify with gel filtration chromatography, polysaccharide components are obtained with molecular weights of about 10⁵ Da to about 10⁷ Da.

During the extraction process, HPLC for purity analysis, UV and infrared spectroscopic identification for qualitative examination, measurement of sugar and glycuronic acid contents respectively by vitriol-phenol and vitriol-carbazole methods, and measurement of monosaccharide compositions of the polysaccharides of different molecular weights and their component ratio with chromatographic techniques and gas chromatography are performed.

Link the above-mentioned polysaccharides (including those prepared from natural gums) with 5-FU, and the linkage method can be acetylating the aforementioned polysaccharides first, and then connecting them with 5-FU under different conditions as per Implementation Example 1; and can also be acetylating 5-FU first, and then connect it with the aforementioned polysaccharides under different conditions as per Implementation Examples 2 and 3.

This method includes connecting 5-FU with the hydroxyl group of polysaccharides through derivation to form an ester or ether linkage, or chemically linking polysaccharides with the —NH part of the 5-FU to form an acylamide linkage through derivation. The formation of the ester linkage is carried out through acyl chloride method or N,N′-dicyclohexylcarbodiimide (DCC) method. The forming of the ether linkage is carried out through condensation, and the formation of the acylamide linkage is derived from aminolysis of acyl chloride.

### ILLUSTRATIVE EMBODIMENTS

In view of the foregoing disclosure several embodiments of the prodrug and its methods of preparation are apparent. The following embodiments are presented for illustrative purposes only and are not meant to limit the scope of the claimed subject matter. Persons of ordinary skill in the art may be able to describe further embodiments based on the guidance set forth in the foregoing disclosure, the examples below and knowledge in the art.

A desirable embodiment is an anti-cancer prodrug with target specificity against galectin-3 expressing cancers and the methodology for preparing the prodrug. The prodrug is synthesized by chemically linking a uniquely prepared polysaccharide with 5-FU through various bridge links.

An additional embodiment is illustrated by a prodrug for the targeted treatment of galectin-3 expressing cancers and the prodrug's method of preparation. For example, the polysaccharide used in preparing the prodrug contains galactose residues.

It is also desirable to provide embodiments of a prodrug for the targeted treatment of galectin-3 expressing cancers wherein the galactose-containing polysaccharide is prepared from natural gums or plant material. These embodiments of the prodrug may have a galactose-containing polysaccharide prepared from pectin, guar gum, and carob bean gum, and the plant materials aloe, medlar and rhubarb. However, virtually any plant material having galactose-containing polysaccharides would make a suitable starting material for isolating said galactose-containing polysaccharide.

The embodiments of the prodrug for targeting galectin-3 expressing cancers may employ have the parent compound 5-FU directly or indirectly linked to a galactose-containing polysaccharide. An indirect linkage is defined as a linkage between 5-FU and a galactose-containing polysaccharide that is mediated by a bifunctional cross-linker. Direct linkages do not employ a linking agent; instead an unmodified or derivatized galactose-containing polysaccharide bonds directly to 5-FU. In other embodiments of the methods for preparing the prodrug the 5-FU may be derivatized. It is understood that derivatizing as referred to herein describes the addition of reactive groups to a galactose-containing polysaccharide or a 5-FU molecule without introducing the spacer arms that characterize commercially available cross-linkers.

Whether the linking is direct or indirect, the prodrug possesses the structural formula Polysaccharides-R-5-FU, in which the R is a linking group where R can comprise any of the following functional groups: —(CH₂)ₙ—, —CO—, —CO(CH₂)ₙ, —CO(CH₂)ₙCO—, and n=1, 2, 3, or 4.

An embodiment of the prodrug may e.g., result from forming a covalent linkage between the 5-FU and free hydroxyl groups of the galactose residues in the polysaccharides. This linkage may be achieved via the formation of ester or ether linkages through derivitization. An illustrative example would be to form the bond between the —NH of 5-FU at the free hydroxyl groups of the galactose residues in the polysaccharides via the formation of an acylamide linkage through derivation.

Embodiments of the methods for preparing the prodrug may use as starting material for galactose-containing polysaccharide isolation, pectin, guar gum, or carob bean gum. Either material is first hydrolyzed with alkali (pH=9-10) then with acid (pH=3−5), and followed by precipitation with alcohol and dialysis. These methods yield galactose-containing polysaccharides of molecular weights from approximately 10⁵Da to approximately 10⁷ Da.

Additional embodiments of the methods for preparing the prodrug may comprise isolating the galactose-containing polysaccharides from aloe, medlar or rhubarb as follows: pulverizing the aloe/medlar/rhubarb plant material, and boiling with ethanol for three eight-hour-periods. The components dissolved in ethanol are extracted. The ethanol insoluble residue is boiled with water for another three eight-hour-periods in order to extract polysaccharides. All the water extractions are finally collected. The polysaccharide-enriched fractions are obtained by precipitation with 5 volumes of ethanol for 3 times. After removing proteins, dialysis, separate and purify with gel filtration chromatography, polysaccharide components are obtained with molecular weights of 10⁵-10⁷ Da. During the extraction process, high-performance liquid chromatography (HPLC) for purity analysis, ultraviolet (UV) and infrared spectroscopic identification for qualitative examination, measurement of sugar and glycuronic acid contents respectively by vitriol-phenol and vitriol-carbazole methods, and measurement of monose compositions of the polysaccharides of different (weight-average) molecular weights and their component ratio with chromatographic techniques and gas chromatography are performed.

An embodiment of the linking methods for linking a galactose-containing polysaccharide and 5-FU is by forming an ester linkage through acyl chloride method or N,N′-dicyclohexylcarbodiimide (DCC) method, the forming of the ether linkage is carried out through condensation, and the formation of the acylamide linkage is derived from aminolysis of acyl chloride.

An additional embodiment of a method for preparing the prodrug may result from forming a covalent linkage between the 5-FU and free hydroxyl groups of the galactose residues in the polysaccharides. This linkage may be achieved via the formation of ester or ether linkages through derivatization. An illustrative example would be to form the bond between the —NH of 5-FU at the free hydroxyl groups of the galactose residues in the polysaccharides via the formation of an acyl amine linkage derived form aminolysis of the acyl chloride.

The embodiments of the prodrug illustrated above are effective for the treatment of galectin-3 expressing cancers, including but not limited to the following: breast, lung, prostate, bladder, thyroid, other head and neck, lymphoma, colon, pancreas and other gastrointestinal cancers.

The examples described below provide illustrative embodiments of methods of preparing the inventive prodrug. It should be readily appreciated that these examples taken together with knowledge in the art would allow persons in the art to practice related embodiments that are clearly encompassed by the subject matter disclosed and claimed herein.

### EXAMPLES

### Example 1

Add 1.2 g of pectin into 52.5 g (0.56 mmol) of melting chloroacetic acid and stir it in solution under 70° C. constant temperature, and then add 35 ml of acetic anhydride. Stir it for 3 hr at a constant temperature of 70° C., pour the solution into a large amount of ice water, forming a yellow precipitate. Separate out the yellow gel-like precipitate, wash it thoroughly with water and ethanol respectively in sequence, collect the precipitate by filtration, and dry it under vacuum at 40° C. for 24 hr to obtain a grayish yellow powder of chloroacetyl pectin.

Weigh 0.38 g of this chloroacetyl pectin and add into 20 ml of dimethyl sulfoxide (DMSO), stir it under 60° C. until it is dissolved. Then put a mixture of 0.65 g of 5-FU and triethylamine into the above-mentioned solution, stir it for 24 hr under 60° C. constant temperature, and, then pour the solution into 100 ml of anhydrous mixture ethanol-ether (1:1 ratio) to produce a loose fluffy precipitation. Let it stand still thoroughly, filter it by vacuum, wash it thoroughly with anhydrous ethanol, and dry it under vacuum at 40° C. for 24 hr to obtain a light yellow precipitate of Pectin-5-FU.

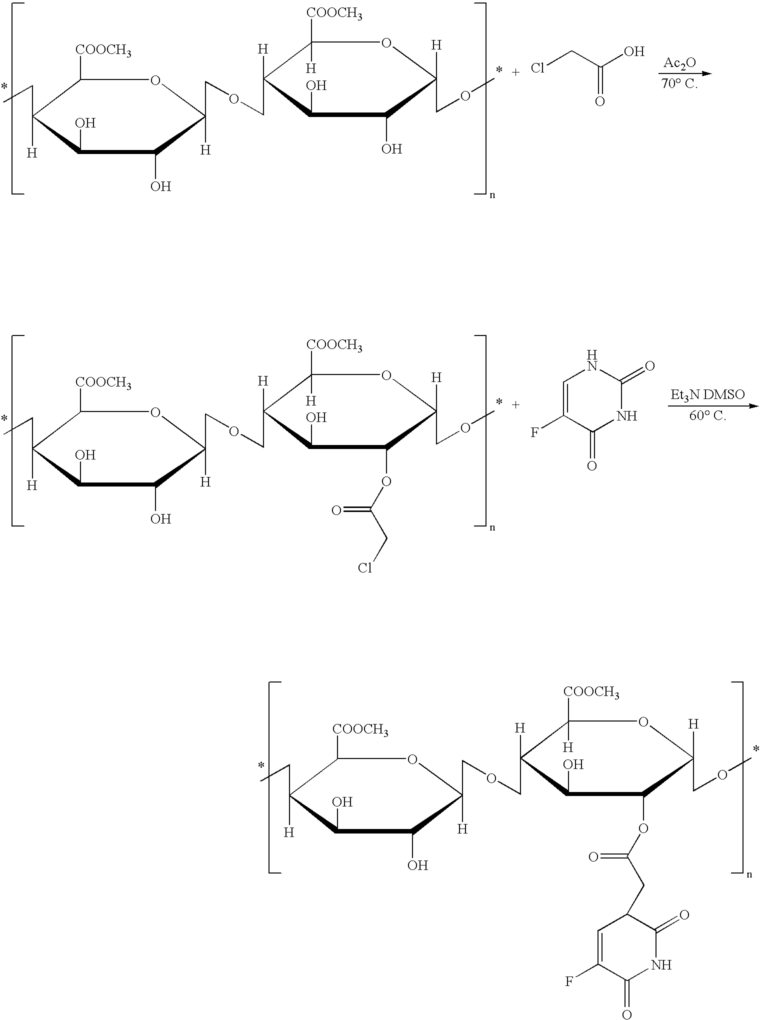

### Example 2

Dissolve 3.92 g of 5-FU and 3.65 g of sodium hydroxide (NaOH) in 22 ml of water, add 12 ml of aqueous solution of 3.30 g of chloroacetic acid, maintain at pH 10, reflux for 2 hr, acidify solution using concentrated HCl to obtain a light brown precipitate. Recrystallize it to obtain 2.26 g of white solid with a yield of ˜40%.

Dissolve 0.5 g of carob bean gum in 20 ml of DMSO, add 0.25 g of N,N′-dicyclohexylcarbodiimide (DCC) and 15 mg of 4-dimethylaminopyridine (DMAP), and then add 0.5 g of 5-FU-1-acetic acid, stirring for 24 hr at 40° C. At completion, pour the reaction mixture into ethanol forming a jelly-like substance. Filter off the jelly-like substance, rinse it with methanol, and then dry under vacuum to obtain final product.

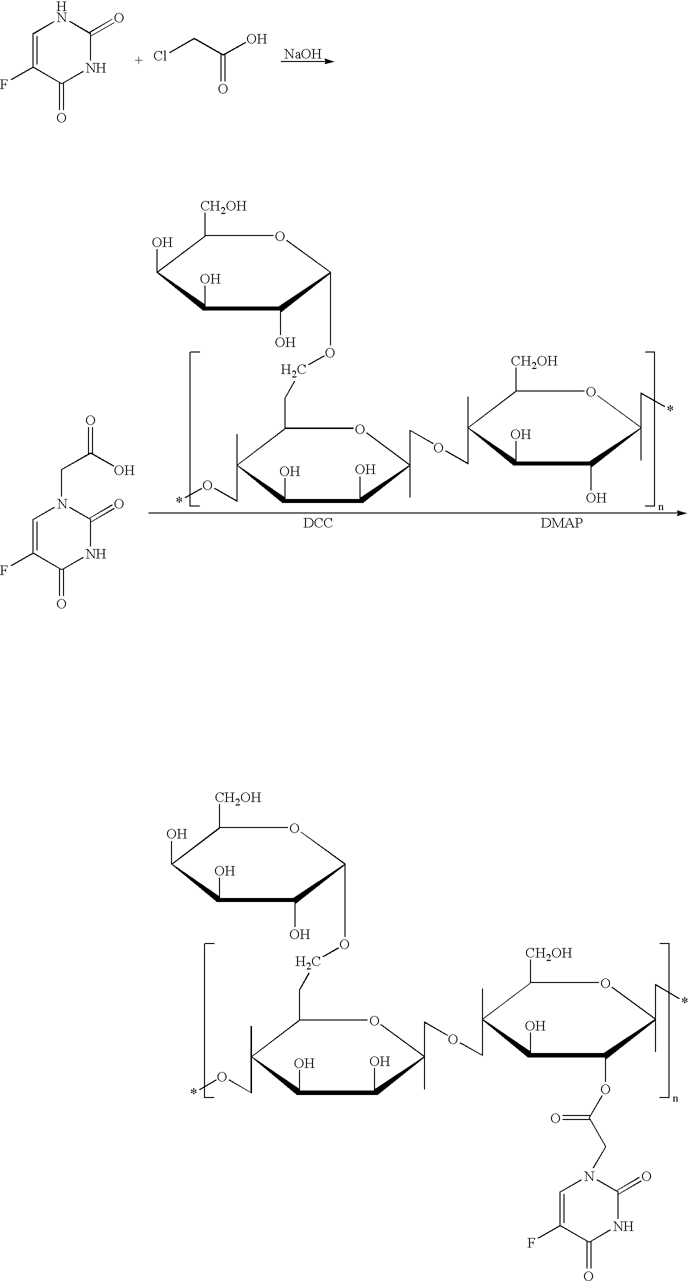

### Example 3

Add 1.0 g of 5-FU in 20 ml of pyridine and stir thoroughly to dissolve the contents into solution. Cool it down to 0° C. in an ice water bath. Add 2 ml of trichloromethyl chloroformate (TCF) slowly dropwise into this 5-FU pyridine solution over 30 minutes. Stir reaction continuously for 1 hr. Remove reaction mixture from the ice water bath. While continuously stirring, allow reaction mixture to warm to room temperature over 2 hr, and then heat reaction mixture to 40° C. and let reaction continue for 30 minutes. Reduce the pressure to remove the unreacted phosgene and pyridine to obtain the brown solid product of chloroformyl 5-FU. Rinse the product with tetrahydrofuran (THF), filter it by vacuum, and dry it by vacuum drying for 6 hr.

Weigh 1 g of guar gum and dissolve it in 20 ml of DMSO. Add 5 ml of pyridine, stir and heat mixture to 40° C. Allow the contents to be dissolved thoroughly, add the chloroformyl 5-FU, stir continuously at room temperature for 24 hr, then heat to 40° C. and allow reaction to continue for 16 hr. The product is precipitated by adding excess anhydrous ethanol-ether (1:1 ratio) and filtered under vacuum. The precipitate is then re-dissolved in DMSO, precipitated with anhydrous ethanol-ether (1:1 ratio), and vacuum filtered. Repeat for two more times to obtain a brown solid product pectin-CH₂—CO-5-FU, then dry under vacuum for 24 hr.

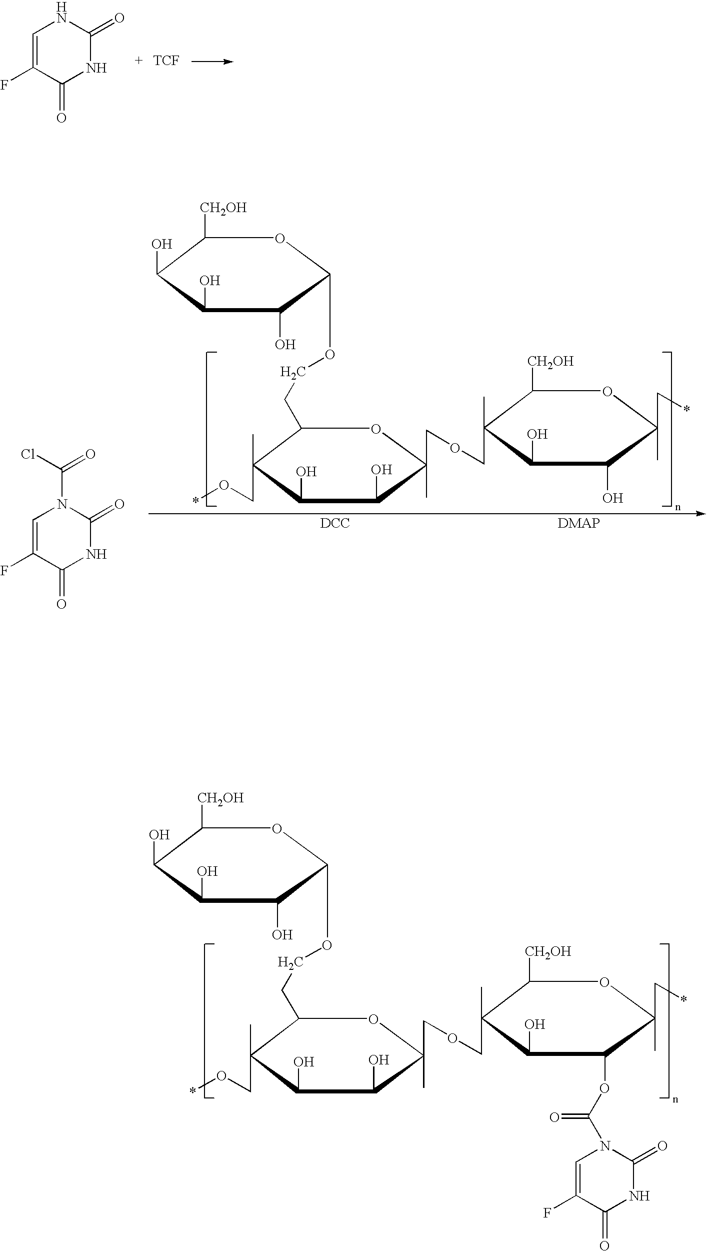

### Example 4

Mix up 5 g of malonic acid, 3 g of benzyl alcohol, 20 ml of toluene, and 100 mg of p-toluene-sulphonic acid (TsOH), and stir it thoroughly, heating to 120° C., and reflux it with a water separator for 1 hr to remove the water. Dissolve the residue in ethyl acetate (60 ml), and then wash with saturated NaCl brine (15 ml) three times. Using a separatory funnel, extract the reaction product from the organic layer (ethyl acetate) first with 1M NaOH (30 ml), then with saturated NaHCO₃ (10 ml), saving both aqueous layers. Repeat this extraction sequence again, saving both aqueous layers. After combining the aqueous layers, add 20 ml of chloroform, and add concentrated hydrochloric acid slowly dropwise while stirring until the water layer is not turbid. Separate and save the organic layer (chloroform), and then extract from the aqueous layer with 10 ml of chloroform twice, saving the organic layers. Combine the extracted chloroform layers and wash them with 10 ml of water twice. Dry them using anhydrous sodium sulfate. Distill off the chloroform by vacuum evaporation and concentrate them to obtain 2.3 g of an oily yellow substance of benzyl malonic acid (compound 1).

Add 0.78 g of 5-FU into 5 ml of hexamethyl aminosilane and heat to 145° C. Add trimethyl chlorosilane slowly dropwise, stir and reflux it for 4 hr, and then distill off the excess hexamethyl aminosilane to obtain the clear, colorless crystal of 2,4-bis(trimethylsilaneoxy)-5-FU (compound 2) for the next reaction step.

Add 2.3 g of compound (1) into 8 ml of thionyl chloride. After stirring and refluxing it for 3 hr at 60° C., distill off the excess thionyl chloride by vacuum evaporation to obtain 2-benzyloxycarbonyl-acetyl chloride. Dissolve this compound into 8 ml of anhydrous acetonitrile, then add it to the above-mentioned 2,4-bis(trimethylsilaneoxy)-5-FU under nitrogen atmosphere. Add 1.68 ml of triethylamine, and reflux for 4 hr at 75° C. Distill off the solvent by vacuum evaporation to obtain a pale brown solid. Recrystallize it in toluene twice to obtain 0.7 g of a white solid, which is 3-(5′-fluoro-3′H-2,′4′-pyridine diketone)-3-oxo-benzyl propionate (compound 3).

Dissolve 0.7 g of compound (3) in anhydrous tetrahydrofuran (THF), and add 10% palladium-carbon mixture (Pd-C), stir at room temperature and bubble with hydrogen gas at atmospheric pressure for 24 hr. Filter the reaction product and concentrate it by vacuum evaporation to obtain 0.5 g of a white solid, which is 3-(5′-fluoro-3′H-2,′4′-pyridine diketone)-3-oxo propionic acid (compound 4). Weigh 0.5 g of aloe polysaccharide (or 0.6 g of medlar polysaccharide or 0.9 g of rhubarb polysaccharide) and dissolve in 20 ml of DMSO, add 0.25 g of N,N′-dicyclohexylcarbodiimide (DCC) and 15 mg of 4-dimethylaminopyridine (DMAP), and 2.5 g of compound (4). Stir the contents at 35° C. for 48 hr. Pour the reaction mixture into ethanol forming a jelly-like substance. Filter the jelly-like substance, rinse it with methanol, and then dry under vacuum to obtain final product.

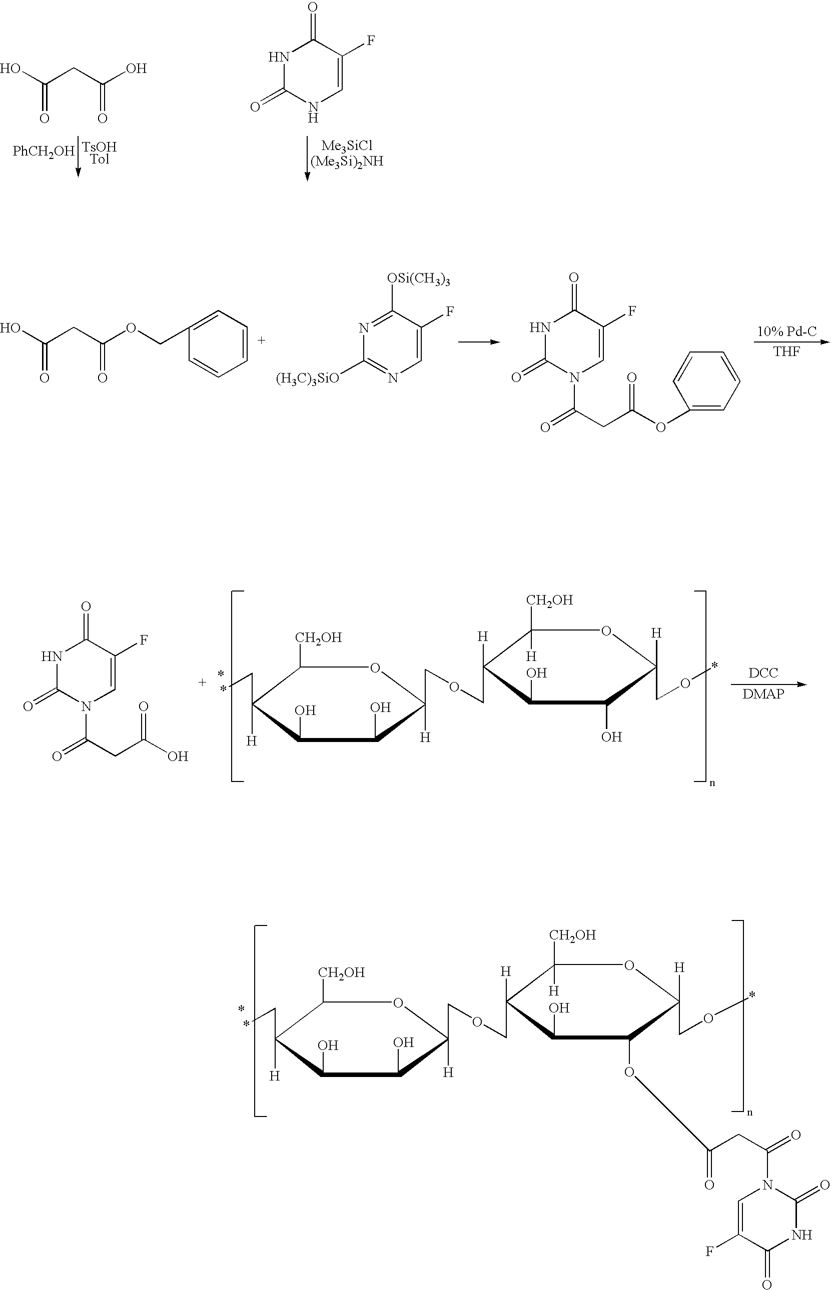

The above linkage method can be substituted using other dicarboxylic acids, where the malonic acid (—COCH₂CO) is substituted with succinic acid —CO(CH₂)₂CO—, glutaric-CO(CH₂)₃CO—, or adipic acid —CO(CH₂)₄CO—, etc.

This invention is not limited to the implementation examples as described in these specifications. The implementation examples are for illustration only. The actual pharmaceutical forms of this invention can be any suitable pharmaceutical formulation in any vehicle to be used for cancer patients.

The examples described below provide illustrative embodiments of methods of preparing numerous embodiments of the inventive prodrug. It should be readily appreciated that these examples taken together with knowledge in the art would allow persons in the art to practice related embodiments that are clearly encompassed by the subject matter disclosed and claimed herein. For example, modified methods of linking Z with a galactose-containing polysaccharide may be encompassed within this disclosure. In addition, many different therapeutic parent compounds may be used as an embodiment of Z.

### EXAMPLES

### Example 1

### Anti-Cancer Drugs

A prodrug with target specificity against colorectal cancer and its preparation methods. Besides the illustrative example of the synthesis of 5-FU prodrug above, other commonly used anti-cancer agents for colorectal cancers such as irinotecan, capecitabine, and camptothecin can employ the same technique to produce novel prodrugs for galectin-3 expressed tumors. The major characteristic of this type of novel compound is that it is a prodrug synthesized by chemically linking a uniquely prepared polysaccharide with 5-fluorouracil (5-FU), irinotecan, capecitabine, and camptothecin through various bridge links. The R group in the following examples is galactose residues with linker groups.

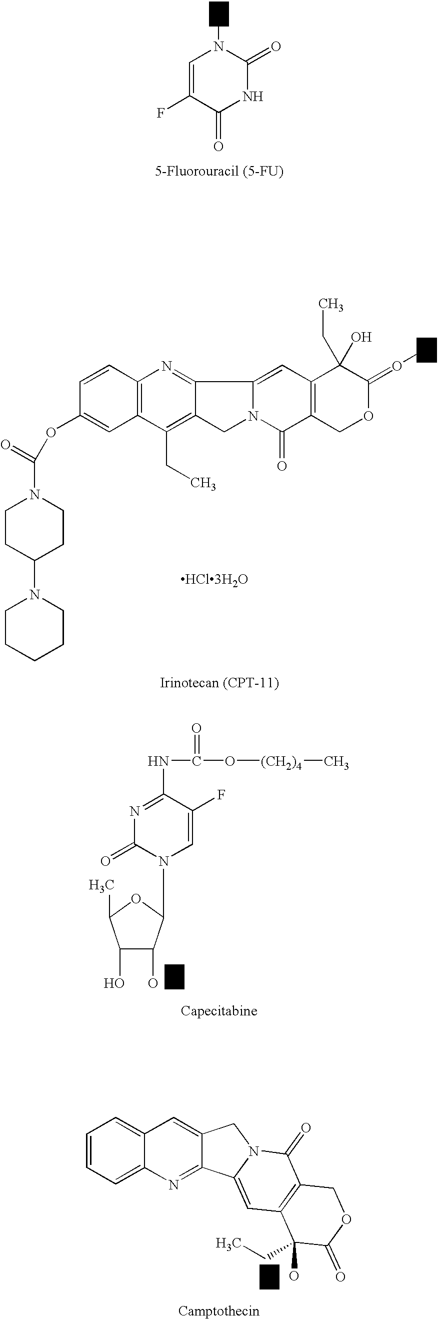

This invention is not limited to the implementation examples as described in these specifications. The implementation examples are for illustration only. The actual pharmaceutical forms of this invention can be any suitable formulation in any vehicle to be used for patients.

Without intent to limit the scope of the invention, exemplary methods and their related results according to the embodiments of the present invention are given above. Note that titles or subtitles are used in the examples for convenience of a reader, which in no way should limit the scope of the invention. Moreover, certain theories are proposed and disclosed herein; however, in no way they, whether they are right or wrong, should limit the scope of the invention.

### REFERENCES

1. Fan C L. Mai C S. Xu J J. Zhuo R X. Synthesis of Condensation Polymers of 1,3-Dihydroxylalkyl-5-Fluorouracil with Dichlorophsphates. Polymer Communications 1985; 1: 77-80. (Publication in Chinese)
2. Luo Y. Zhuo R X. Fan C L. Studies on the Synthesis and Antitumor Activity of Biodegradable Polyphosphamides. Chemical Journal of Chinese Universities 1994; 15: 767-770. (Publication in Chinese)
3. Ohya Y. Inosaka K. Ouchi T. Synthesis and antitumor activity of 6-O-carboxymethyl chitin fixing 5-fluorouracils through pentamethylene, monomethylene spacer groups via amide, ester bonds. Chemical & Pharmaceutical Bulletin 1992; 40: 559-61.
4. Ohya Y. Takei T. Kobayashi H. Ouchi T. Release behavior of 5-fluorouracil from chitosan-gel microspheres immobilizing 5-fluorouracil derivative coated with polysaccharides and their cell specific recognition. Journal of Microencapsulation 1993; 10:1-9.
5. Ouchi T. Hagihara Y. Takahashi K. Takano Y. Igarashi I. Synthesis and antitumor activity of poly(ethylene glycol)s linked to 5-fluorouracil via a urethane or urea bond. Drug Design & Discovery 1992; 9:93-105.
6. Schoeppner H L. Raz A. Ho S B. Bresalier R S. Expression of an endogenous galactose-binding lectin correlates with neoplastic progression in the colon. Cancer 1995; 75: 2818-26.
7. Yoshii T. Fukumori T. Honjo Y. Inohara H. Kim H R C. Raz A. Galectin-3 phosphorylation is required for its anti-apoptotic function and cell cycle arrest. Journal of Biological Chemistry 2002; 277: 6852-6857.
8. Zhu K J. Zhang J X. Wang C. Yasuda H. Ichimaru A. Yamamoto K. Preparation and in vitro release behaviour of 5-fluorouracil-loaded microspheres based on poly(L-lactide) and its carbonate copolymers. Journal of Microencapsulation 2003; 20: 731-43.
9. Buttery R, Monaghan H, Salter D, Sethi T. Galectin-3: Differential Expression between Small-Cell and Non-Small-Cell Lung Cancer. Histopathology 2004; 44: 339-44.
10. Califice S, Castronovo V, van den Brule F. Galectin-3 and Cancer (Review). International J Oncol 2004; 25: 983-992.
11. Le Marer N, Hughes R. Effects of the Carbohydrate-Binding Protein Galectin-3 on the Invasiveness of Human Breast Carcinoma Cells. J Cell Physiol 1996; 168: 51-8.
12. Moon B, Lee Y, Battle P, Jessup J, Raz A, Kim H. Galectin-3 Protects Human Breast Carcinoma Cells against Nitric Oxide-Induced Apoptosis Implication of Galectin-3 Function during Metastasis. Am J of Pathology 2001; 159:1055-1060.
13. Perillo N, Marcus M, Baum L. Galectins: Versatile Modulators of Cell Adhesion, Cell Proliferation, And Cell Death. J Molecular Med 1998; 76: 402-412.
14. Takenaka Y, Fukumori T, Raz A. Galectin-3 and Metastasis. Glycoconjugate Journal 2002; 19: 543-549.
15. Teymoortash A, Pientka A, Schrader C, Tiemann M, Werner J. Expression of Galectin-3 in Adenoid Cystic Carcinoma of the Head and Neck and its Relationship with Distant Metastasis. J Cancer Res & Clin Oncol 2006; 132:51-56
16. Van den Brule F, Califice S, Castronovo V. Expression of Galectins in Cancer: A Critical Review. Glycoconjugate Journal 2002; 19: 537-542.

U.S. Patent Documents

| | | |
|---|---|---|
| 4,605,738 | Aug. 12, 1986 | Kamata, et al. |
| 4,622,325 | Nov. 11, 1986 | Fujii, et al. |
| 4,631,342 | Dec. 23, 1986 | Umemoto, et al. |
| 4,650,801 | Mar. 17, 1987 | Fujii, et al. |
| 4,652,570 | Mar. 24, 1987 | Fujii, et al. |
| 4,704,393 | Nov. 3, 1987 | Wakabayashi, et al. |
| 4,719,213 | Jan. 12, 1988 | Fujii, et al. |
| 4,757,139 | Jul. 12, 1988 | Kawaguchi, et al. |
| 4,810,790 | Mar. 7, 1989 | Yamamoto, et al. |
| 4,864,021 | Sep. 5, 1989 | Fujii |
| 4,914,105 | Apr. 3, 1990 | Fujii, et al. |
| 4,983,609 | Jan. 8, 1991 | Fujii |
| 5,032,680 | Jul. 16, 1991 | Kawai, et al. |
| 5,047,521 | Sep. 10, 1991 | Fujii, et al. |
| 5,049,551 | Sep. 17, 1991 | Koda, et al. |
| 5,077,055 | Dec. 31, 1991 | Muller, et al. |
| 5,089,503 | Feb. 18, 1992 | Johnson |
| 5,116,600 | May 26, 1992 | Fujii, et al. |
| 5,457,187 | Oct. 10, 1995 | Gmeiner, et al. |
| 5,496,810 | Mar. 5, 1996 | Schwartz |
| 5,610,160 | Mar. 11, 1997 | Sloan, et al. |
| 5,614,505 | Mar. 25, 1997 | Gmeiner, et al. |
| 5,627,187 | May 6, 1997 | Katz |
| 5,663,321 | Sep. 2, 1997 | Gmeiner, et al. |
| 5,676,973 | Oct. 14, 1997 | Levin |
| 5,808,049 | Sep. 15, 1998 | Yamazaki, et al. |
| 5,817,666 | Oct. 6, 1998 | Katz |
| 5,843,917 | Dec. 1, 1998 | Boyd, et al. |
| 6,403,569 | Jun. 11, 2002 | Achterrath |
| 6,670,335 | Dec. 30, 2003 | Singh, et al. |
| 6,794,370 | Sep. 21, 2004 | Achterrath |

Foreign Patent Documents

| | | |
|---|---|---|
| 1246204 | October, 1989 | JP. |
| 2091009 | March, 1990 | JP. |

What is claimed is:

1. A prodrug suitable for targeted delivery of a therapeutic compound to a tumor expressing galectin-3, comprising,
a) a polysaccharide bound by galectin-3;
b) a parent therapeutic compound, and
c) a covalent bond connecting a) to b).

2. The prodrug of claim 1 wherein the polysaccharide is a galactose-containing polysaccharide.

3. The prodrug of claim 1 wherein the polysaccharide comprises one or more galactose residues available for binding to galectin-3.

4. The prodrug of claim 2 or 3 wherein the galactose-containing polysaccharide has a molecular weight of about 10⁵ Da to about 10⁷ Da.

5. The prodrug of claim 1 wherein the therapeutic parent compound comprises at least one atom available to form a covalent linkage with the galactose-containing polysaccharide, the atom being oxygen, nitrogen or sulfur.

6. A prodrug having the structural formula polysaccharide-R-Z, wherein Z comprises a therapeutic parent compound and R comprises a covalent bond between Z and the polysaccharide, and wherein the polysaccharide is a galactose-containing polysaccharide.

7. The prodrug of claim 5, wherein R comprises either an ester, an ether, an amide, an amine, a hydroxylamine, a thioether or thioester.

8. The prodrug of claim 1, wherein the galactose-containing polysaccharide occurs naturally.

9. The prodrug of claim 1, wherein the galactose-containing polysaccharide and the therapeutic parent compound are linked by a covalent bond comprising a linkage selected from the group consisting of —(CH₂)ₙ—, —CO—, —CO(CH₂)ₙ—, and —CO(CH₂)ₙ—CO— and wherein n is from 1 to 4.

10. The prodrug of claim 1 or 5, wherein the galactose-containing polysaccharide, or a galactose-containing fragment thereof, is capable of binding to galectin-3.

11. The prodrug of claim 1 wherein the prodrug has the structure shown in FIG. 1.

12. The prodrug of claim 10 comprising at least one galactose-containing fragment to which the therapeutic parental compound is covalently linked.

13. The prodrug of claim 10, wherein the at least one galactose-containing fragment results from the action of bacterial enzymes that degrade the galactose-containing polysaccharide.

14. The prodrug of claim 13, wherein the galactose-containing fragment further comprises the parental therapeutic compound.

15. The prodrug of claim 13, wherein the bacterial enzymes that produce the galactose-containing fragment are in the colon.

16. The prodrug of claim 5 wherein Z is 5-fluorouracil (5-FU), irinotecan, capecitabine, or camptothecin.

17. A method for preparing a prodrug having affinity for galectin-3, the structural formula being polysaccharide-R-Z, wherein
a) Z comprises a parent compound and R comprises a covalent bond connecting Z to the polysaccharide, and wherein the polysaccharide is a galactose-containing polysaccharide, the method comprising the steps of;
b) hydrolyzing pectin, guar gum and carob bean gum in alkali at a pH from about 9 to about 10;
c) hydrolyzing the product of step a) in acid at a pH from about 3 to about 5; and
d) purifying the galactose-containing polysaccharide, and reacting the galactose-containing polysaccharide with a parent therapeutic compound Z, thereby forming covalent bond R comprising either an ester, an ether, an amide, an amine, an acyl amine a hydroxylamine, a thioester, or a thioether.

18. A method for preparing a prodrug having the structural formula
a) polysaccharide-R-Z, comprising the steps of,
b) pulverizing either aloe, medlar, or rhubarb and treating the pulverized material with ethanol to obtain a soluble phase and an insoluble residue;
c) extracting the insoluble residue in boiling water to obtain polysaccharides,
d) purifying the polysaccharide, and
e) reacting the polysaccharide with a therapeutic parent compound Z to form covalent bond R comprising either an ester, an ether, an amide, an amine, an acyl amine, a hydroxylamine, a thioester, or a thioether.

19. The method of claim 17 or 18 further comprising derivatizing the polysaccharide so as to add a functional group from the group consisting of an ester, an ether, an amide, an amine, a hydroxylamine, a thioether and a thioester.

20. The method of claim 19 wherein the added functional group forms a covalent bond with the parent compound, and the covalent bond comprises a linkage selected from the group consisting of —(CH₂)ₙ—, —CO—, —CO(CH₂)ₙ—, and CO(CH₂)ₙ—CO—, wherein n is from 1 to 4.

21. The method of claim 21 or 22 wherein Z is 5-fluorouracil (5-FU), irinotecan, capecitabine, or camptothecin.

22. A pharmaceutical composition comprising an effective amount of a prodrug having the structural formula polysaccharide —R-Z, comprising
a) A naturally occurring galactose-containing polysaccharide
b) Z comprises a therapeutic parent compound and
c) R comprises a covalent bond connecting Z to the polysaccharide,
and a pharmaceutically suitable carrier, filler or adjuvant.

23. A pharmaceutical composition comprising an effective amount of a prodrug having the structural formula polysaccharide —R-Z, comprising
a) a naturally occurring galactose-containing polysaccharide
b) Z comprises 5-fluorouracil (5-FU), irinotecan, capecitabine, or camptothecin. and
c) R comprises a —(CH₂)ₙ—, —CO—, —CO(CH₂)ₙ—, and —CO(CH₂)ₙ—CO—, wherein n is from 1 to 4. and a pharmaceutically suitable carrier, filler or adjuvant.

24. The pharmaceutical composition of claim 23 wherein Z is 5-FU and R comprises a —(CH₂)ₙ—, —CO—, —CO(CH₂)ₙ—, and —CO(CH₂)ₙ—CO— wherein n is from 1 to 4.

25. The pharmaceutical composition of claim 23 wherein Z is 5-FU and R comprises a —CO(CH₂)ₙ—CO—, and wherein n is from 1 to 4.

26. The pharmaceutical composition of claim 23 wherein Z is 5-FU and R comprises a —CO—.

27. A method for treating a galectin-3-expressing tumor comprising the step of,
a) providing a prodrug of the structural formula polysaccharide —R-Z.
b) administering an effective amount of the prodrug to a subject in need thereof,
c) wherein Z is anticancer compound and
d) the polysaccharide comprises a galactose-containing polysaccharide having at least one galactose suitable for binding to galectin-3.

28. The method of claim 27, wherein the anticancer compound is selected from the group consisting of 5-fluorouracil (5-FU), irinotecan, capecitabine, and camptothecin.

29. The method of claim 27, wherein the anticancer compound is 5-fluorouracil (5-FU).

30. The method of claim 27, wherein R comprises an ester, an ether, an amide, an acyl amine or an amine.

31. The method of claim 27, wherein the polysaccharide has a molecular weight of from approximately 10⁵ Da to about 10⁷ Da.

32. The method of claim 27, wherein the effective amount of the prodrug encompasses a pharmaceutical composition further comprising a pharmaceutically suitable adjuvant, filler or carrier.

33. The method of claim 27, wherein the administering is by the oral route.

34. The method of claim 27, wherein the galectin-3 expressing tumor is selected from the group consisting of breast, lung, prostate, bladder, thyroid, head and neck, lymphomas, colorectal, and pancreatic tumors.

35. The method of claim 27, wherein the galectin-3 expressing tumor is a colorectal tumor.

36. The method of claim 27, wherein the galactose-containing polysaccharide is isolated from guar gum, carob bean gum, aloe, medlar or rhubarb.

37. The method of claim 27, wherein galactose-containing polysaccharide is pectin.
